(19) United States
(12) Patent Application Publication
(10) Pub. No.: US 2004/0122107 A1
(43) Pub. Date: Jun. 24, 2004

(54) Method of alleviating barometric-induced symptoms in airline passengers

(76) Inventor: Jeffrey R. Fine, Charlestown, MA (US)

Correspondence Address: MIRICK O'CONNELL, 1700 WEST PARK DRIVE, WESTBOROUGH, MA, 01581-3941, US

(21) Appl. No.: 10/662,137

(22) Filed: Sep. 12, 2003

Related U.S. Application Data
(60) Provisional application No. 60/410,633, filed on Sep. 13, 2002.

(51) Int. Cl. A61K 31/137

(52) U.S. Cl. 514/649

(57) ABSTRACT

A method of alleviating the symptoms of ear and sinus cavity blockage in a descending aircraft, and a kit that provides the medications and instructions for a user to accomplish the method. The method contemplates ingesting a nasal decongestant at least one hour before the scheduled aircraft landing time, for non-specific shrinking of the nasal lining, and applying a nasal decongestant spray into the nose later in flight than the ingestion of the nasal decongestant, to shrink the nasal lining. The ingested and sprayed decongestants help to shrink the mucosa, including at least the nasal lining, to decrease the pain associated with blockage as an aircraft descends. The ingested nasal decongestant currently comprises pseudoephedrine, and the preferred dose is about 60 mg. The sprayed nasal decongestant currently comprises either phenylephrine or oxymetazoline, either of which may be in a concentration up to about 1%.

### CROSS REFERENCE TO RELATED APPLICATION

This application claims priority of Provisional application serial No. 60/410,633, filed on Sep. 13, 2002.

### FIELD OF THE INVENTION

This invention relates to a method and kit for alleviating sinus and ear pain associated with landing in a commercial aircraft.

### BACKGROUND OF THE INVENTION

According to NBTA (FAA) estimates there were about 700 million emplanements in the U.S. alone in 2001. Factor in the Academy of Allergy, Asthma, and Immunology estimate that 20% of the population is affected by allergic diseases and incorporate the assumption that, at any given time, 5% of the population have a “cold” or are otherwise irritated (affected) by acknowledged poor air quality on aircraft, then there is the potential for over 170 million people to have significant ears and sinus difficulty while flying. Significant difficulty means the following: There is an intimate relationship between swelling in the nasal passages and potential blockages in the ears and sinus cavities. This typically takes place within the hour before landing (because of the pressurization characteristics of aircraft and the normal descent patterns of commercial aircraft). The most prominent symptoms are blockage of the sinuses. Blockage of the ears occurs when the pressurization of the aircraft drops below eight thousand feet, which usually occurs within about an hour of landing in commercial aircraft. This is the time at which appropriate preemptive treatments will be useful in obviating barometric-induced symptoms of ear blockage, which can range from just blockage to severe pain and actual hemorrhage or rupture of the eardrum. The same barometric scenario which produces blocked ears, also accounts for blocked sinuses and associated pain.

### SUMMARY OF THE INVENTION

There is a significant need for a safe and effective means of obviating these troublesome occurrences.

Accordingly, this invention features a method of alleviating the symptoms of ear and sinus cavity blockage in a descending aircraft, comprising ingesting a nasal decongestant at least one hour before the scheduled aircraft landing time, for non-specific shrinking of the nasal lining, and applying a nasal decongestant spray into the nose later in flight than the ingestion of the nasal decongestant, to shrink the nasal lining. The ingested and sprayed decongestants help to shrink the mucosa, including at least the nasal lining, to decrease the pain associated with blockage as an aircraft descends. The ingested nasal decongestant currently comprises pseudoephedrine, and the preferred dose is about 60 mg. The sprayed nasal decongestant currently comprises either phenylephrine or oxymetazoline, either of which may be in about a 1% concentration.

The ingestion step preferably takes place within about six hours of the scheduled aircraft landing time, and the spray step preferably takes place within about one hour of the scheduled aircraft landing time.

In a more specific embodiment, the invention features a method of alleviating the symptoms of ear and sinus cavity blockage in a descending aircraft, comprising ingesting about 60 mg of pseudoephedrine at least one hour before the scheduled aircraft landing time, for non-specific shrinking of the nasal lining, and applying a nasal decongestant spray into the nose after the pseudoephedrine ingestion and within about one hour of scheduled landing time, to shrink the nasal lining.

This invention also features a kit for use in alleviating the symptoms of ear and sinus cavity blockage in descending aircraft, comprising a first medication comprising an oral nasal decongestant and a second medication comprising a nasal spray decongestant. The kit also includes instructions for the user to ingest a proper dose of the oral nasal decongestant at least one hour before the scheduled aircraft landing time, and to subsequently spray the nasal spray decongestant into at least one nostril within about one hour of the scheduled landing time.

### DETAILED DESCRIPTION OF THE PREFERRED EMBODIMENTS

The invention is designed to overcome the major issues of what is called Eustachian tube and sinus blockage during an aircraft flight. It consists of a first medication, an oral nasal decongestant medication, taken as the flight starts if the flight is less than six hours. For longer flights, the oral medication is preferably taken about six hours before the scheduled landing time. A second medication, a nasal decongestant spray, is taken close to landing, typically within about one hour of the scheduled landing time. Included with the appropriate medication in a kit will be instructions for dosage and timing thereof, and medical information about safe usage and contraindications to usage. The invention preferably includes either two 60 mg. pseudoephedrine (e.g., Sudafed™) or four 30 mg. pseudoephedrine (e.g., Sudafed™) capsules, and one spray bottle of phenylephrine (e.g., Neosynephrine™) or oxymetazoline (e.g., Afrin™) at an appropriate typical dose, typically up to about 1% concentration.

More details about the composition of the invention, pharmacologically, are discussed below. The first (early in flight) medication is a nasal decongestant (called an alpha adrenergic agent) which, when taken orally, serves to shrink the nasal lining (mucosa) nonspecifically. A preferred mediation is pseudoephedrine, and the adult dose is preferably 60 mg. Nonspecifically means it does not make a difference whether the swelling is secondary to an ordinary cold or an allergy. Pseudoephedrine is primarily used for the treatment of “cold” symptoms, but is often included, with an antihistamine, for the treatment of allergic congestion. The rationale for its use is to provide some decrease in normal nasal swelling as the flight progresses. There are continuing allusions to the poor air quality in aircraft. The major manifestations of this “poor quality” are nasal drying, congestion etc., resulting in nasal swelling that is alleviated by the pseudoephedrine.

The second (late in flight) medication is a topical nasal decongestant delivered as a nasal spray, typically one or two sprays into each nostril. This has the topical effect of shrinking the lining of the nose and, ostensibly, the Eustachian tubes, which basically have the same type of tissue lining as the nose. Phenylephrine or oxymetazoline are the preferred choices. This topical is an adjunct to the systemic medication. The timing of the use of the nasal spray is designed to coincide with flight dynamics. The average landing pattern internationally is about an hour. It is during this landing period that most people experience both ear and sinus pressure symptoms. The rationale for the spray is that it defuses that congestion immediately and obviates the associated barometric otitis (ear pressure) and barometric sinus pressure.

The oral medication and the spray medication act adjunctively and additively to relieve the potential for ear and sinus symptoms associated with descent. This is the rationale for combining both the pill and the spray.

The effective substitutes for the sprays are limited. Under the current understanding of sprayable nasal decongestants, one either uses phenylephrine or oxymetazoline. With the withdrawal of phenylopropolyene from the market, the only effective oral (pill) decongestant is pseudoephedrine. In the lexicon, both topical and oral decongestants are the same.

The invention can be presented for sale on a rack of pull-off, sealed plastic pouches. Attached to the rack will be a laminated copy of all pages of the use/safety manual, for perusal before purchase. The product will preferably contain enough mediation for two flights (one round trip). The product will preferably contain the indications and rationale for usage, the method and timing of usage, and the medical contraindications of usage.

Other embodiments will occur to those skilled in the art and are within the following claims:

What is claimed is:

1. A method of alleviating the symptoms of ear and sinus cavity blockage in a descending aircraft, comprising:
ingesting a nasal decongestant at least one hour before the scheduled aircraft landing time, for non-specific shrinking of the nasal lining; and
applying a nasal decongestant spray into the nose later in flight than the ingestion of the nasal decongestant, to shrink the nasal lining;
wherein the ingested and sprayed decongestants help to shrink the mucosa, including at least the nasal lining, to decrease the pain associated with blockage as an aircraft descends.

2. The method of claim 1 wherein the ingested nasal decongestant comprises pseudoephedrine.

3. The method of claim 2 wherein the pseudoephedrine dose is about 60 mg.

4. The method of claim 1 wherein the sprayed nasal decongestant comprises phenylephrine.

5. The method of claim 4 wherein the phenylephrine is in up to about a 1% concentration.

6. The method of claim 1 wherein the sprayed nasal decongestant comprises oxymetazoline.

7. The method of claim 6 wherein the oxymetazoline is in up to about a 1% concentration.

8. The method of claim 1 wherein the ingestion step takes place within about six hours of the scheduled aircraft landing time.

9. The method of claim 1 wherein the spray step takes place within about one hour of the scheduled aircraft landing time.

10. A method of alleviating the symptoms of ear and sinus cavity blockage in a descending aircraft, comprising:
ingesting about 60 mg of pseudoephedrine at least one hour before the scheduled aircraft landing time, for non-specific shrinking of the nasal lining; and
applying a nasal decongestant spray into the nose after the pseudoephedrine ingestion and within about one hour of scheduled landing time, to shrink the nasal lining;
wherein the ingested and sprayed decongestants help to shrink the mucosa, including at least the nasal lining, to decrease the pain associated with blockage as an aircraft descends.

11. The method of claim 10 wherein the sprayed nasal decongestant comprises phenylephrine.

12. The method of claim 1 wherein the sprayed nasal decongestant comprises oxymetazoline.

13. A kit for use in alleviating the symptoms of ear and sinus cavity blockage in descending aircraft, comprising:
a first medication comprising an oral nasal decongestant;
a second medication comprising a nasal spray decongestant; and
instructions for the user to ingest a proper dose of the oral nasal decongestant at least one hour before the scheduled aircraft landing time, and to subsequently spray the nasal spray decongestant into at least one nostril within about one hour of the scheduled landing time;
wherein the ingested and sprayed decongestants help to shrink at least the nasal lining, to decrease pain associated with blockage as the aircraft descends.

14. The kit of claim 13 wherein the ingested nasal decongestant comprises pseudoephedrine.

15. The kit of claim 14 wherein the pseudoephedrine dose is about 60 mg.

16. The kit of claim 13 wherein the sprayed nasal decongestant comprises phenylephrine.

17. The kit of claim 16 wherein the phenylephrine is in up to about a 1% concentration.

18. The kit of claim 13 wherein the sprayed nasal decongestant comprises oxymetazoline.

19. The kit of claim 18 wherein the oxymetazoline is in up to about a 1% concentration.

20. The kit of claim 13 wherein the instructions comprise ingesting the first medication within about six hours of the scheduled aircraft landing time.
